(19) United States
(12) Patent Application Publication
(10) Pub. No.: US 2012/0266325 A1
(43) Pub. Date: Oct. 18, 2012

(54) Plant Stress Tolerance Related Protein GmSIK1 and Encoding Gene and Use Thereof

(76) Inventors: Shouyi Chen, Beijing (CN); Jinsong Zhang, Beijing (CN); Peng Liu, Beijing (CN); Wanke Zhang, Beijing (CN); Sijie He, Beijing (CN); Qing Lin, Beijing (CN)

(21) Appl. No.: 13/503,163

(22) PCT Filed: Oct. 15, 2010
(86) PCT No.: PCT/CN2010/077770

§ 371 Date: Jun. 26, 2012

(30) Foreign Application Priority Data
Oct. 20, 2009 (CN) 200910236141.2

(51) Int. Cl. C07K 14/415; C12N 15/63; A01H 1/06; C12N 5/10; C07H 21/04; C12N 15/29; C12N 1/21

(52) U.S. Cl. 800/278; 530/378; 536/23.6; 435/320.1; 435/252.3; 435/419; 536/24.33

(57) ABSTRACT

Provided are a plant stress tolerance related protein GmSIK1 and encoding gene and use thereof. The GmSIK1 protein has the amino acid sequence as shown in SEQ ID NO: 2. The transgenic plant with enhanced stress tolerance such as drought tolerance and/or salt tolerance can be obtained from introducing the encoding gene of GmSIK1 protein into plant cell.

### FIELD OF THE INVENTION

The present invention relates to a plant stress tolerance related protein GmSIK1, and an encoding gene and a use thereof, and particularly to a stress tolerance related protein GmSIK1 derived from soybean; and an encoding gene and a use thereof.

### BACKGROUND OF THE INVENTION

Growth and development of plants are closely related to the external environment, and the plants will withstand all kinds of stress in each growth and development stage, including biotic and abiotic stress. The abiotic stress such as drought, salinity, and low temperature is an important factor that restricts the growth of plants, and lowers the yield and quality of crops. In agricultural production, due to the crop yield loss caused by abiotic stress, the average yield of major crops in the world is decreased by 50% or higher. After a long time of evolution, the plants have mechanisms to adapt to different stress. One important mechanism is to induce the expression of related genes. The genes implicated in the response to abiotic stress are generally classified into two types. One type is functional genes encoding effector proteins that directly protect the plant cells from stress damage, for example, enzymes involved in biosynthesis of osmoprotectants, late embryogenesis abundant (LEA) proteins, antifreeze proteins, chaperones, detoxification proteins, and aquaporins; and the other type is regulatory genes, products of which regulate gene expression and signaling of plants in response to abiotic stress, including transcription factors, protein kinases and related enzymes involved in phosphoinositide metabolism.

Receptor-like kinases are the largest gene family in plant protein kinase families. Plant receptor-like kinases and cytoplasmic kinases both belong to the receptor-like kinase gene family. The receptor-like kinase family is classified according to a systematic evolutionary relationship between domains of each member kinase. At present, the full-length receptor-like kinase genes in more than 20 flowering plants have been cloned. The plant receptor-like kinases may be classified into two main types based on the functions thereof. One type is those involved in regulation of growth and development of plants under normal growth conditions. The other type is those involved in stress response and interaction between plants and microorganisms.

Knowledge of transduction of stress signals in plants is critical for understanding and regulating the stress tolerance of the plants. The protein kinases and especially the receptor-like kinases play a critical role in signal transduction; however, due to insufficient knowledge of the plant receptor-like kinases, there is a considerable lack of data about the action mechanism thereof. Therefore, the research of the stress tolerance mechanism of the receptor-like kinases is of great theoretical significance.

As a most important oil crop and a main source of plant protein, the improvement of the stress tolerance of soybean is of great theoretical and practical significance.

### SUMMARY OF THE INVENTION

An objective of the present invention is to provide a plant stress tolerance related protein, and an encoding gene thereof.

The protein provided in the present invention is referred to as GmSIK1 (Stress Inducible Kinase 1) and derived from soybean, and is specifically a protein shown in (a), (b), (c) or (d) below:

(a) a protein consisting of amino acid residues 34-714 starting from an N-terminus of an amino acid sequence as shown in SEQ ID NO.: 2 in the sequence list;

(b) a protein consisting of the amino acid sequence as shown in SEQ ID NO.: 2 in the sequence list;

(c) a protein related to plant stress tolerance and derived from (a) by substituting and/or deleting and/or adding one or more amino acid residues in the amino acid sequence as shown in SEQ ID NO.: 2 in the sequence list; and

(d) a protein related to plant stress tolerance and consisting of an amino acid sequence that is at least 75%, at least 80%, at least 85%, at least 90%, at least 95%, at least 96%, at least 97%, at least 98% or at least 99% homologous with the amino acid sequence as shown in SEQ ID NO.: 2 in the sequence list.

SEQ ID NO: 2 in the sequence list consists of 714 amino acid residues, including a signal peptide (amino acid residues 1-33 starting from N-terminus), 3 transmembrane regions (amino acid residues 5-27, 294-313, and 333-355 respectively, starting from N-terminus), and an intracellular kinase domain (amino acid residues 392-662 starting from N-terminus), and has typical features of the structure of the receptor-like kinases. The receptor-like kinase family can be classified into different subfamilies according to an evolutionary relationship of the kinase domains, and according to this classification method, GmSIK1 belongs to the LRK-10L subfamily, which has no known extracellular domain.

The substitution and/or deletion and/or addition of one or more amino acid residues refers to a substitution and/or deletion and/or addition at any position in the sequence mentioned in (a) or (b), and preferably 1-20 amino acid residues are substituted and/or deleted and/or added, more preferably 1-15 amino acid residues are substituted and/or deleted and/or added, more preferably 1-10 amino acid residues are substituted and/or deleted and/or added, and most preferably 1-5 amino acid residues are substituted and/or deleted and/or added.

For ease of purification of the protein mentioned in (a), (b), (c), or (d), a label as shown in Table 1 may be attached to the N-terminus or C-terminus of the protein.

TABLE 1
Sequence of labels

| Label | Residue | Sequence |
|---|---|---|
| Poly-Arg | 5-6 (generally 5) | RRRRR |
| Poly-His | 2-10 (generally 6) | HHHHHH |
| FLAG | 8 | DYKDDDDK |
| Strep-tag II | 8 | WSHPQFEK |
| c-myc | 10 | EQKLISEEDL |

The protein mentioned above in (a), (b), (c) or (d) may be artificially synthesized, or obtained by synthesizing an encoding gene thereof first, and then biologically expressing. The encoding gene of the protein mentioned above in (c) or (d) may be obtained by deleting codons of one or more amino acid residues from the DNA sequence as shown in SEQ ID NO.: 1 in the sequence list, and/or through missense mutation of one or more base pairs, and/or by attaching an encoding sequence of a label shown in Table 1 at a 5′ end and/or a 3′ end.

The substitution, replacement and/or addition of one or more amino acid residues in the amino acid sequence of the protein may be caused by spontaneous mutations or caused by artificial mutagenesis.

The encoding gene provided in the present invention may be specifically a gene shown in 1), 2) or 3) below:

1) a DNA molecule as shown in SEQ ID NO.: 1 in the sequence list;

2) a DNA molecule that specifically hybridizes the DNA molecule defined in 1) under stringent conditions and encodes the plant stress tolerance related protein; and

3) a DNA molecule that is at least 70%, at least 75%, at least 80%, at least 85%, at least 90%, at least 95%, at least 96%, at least 97%, at least 98% or at least 99% homologous with the DNA sequence defined in 1) and encodes the plant stress tolerance related protein.

The specific hybridization conditions may be hybridization at 50° C. in a mixed solution of 7% sodium dodecyl sulfate (SDS), 0.5 M NaPO₄, and 1 mM EDTA, and washing at 50° C. in 2×SSC, and 0.1% SDS; hybridization at 50° C. in a mixed solution of 7% sodium dodecyl sulfate (SDS), 0.5 M NaPO₄, and 1 mM EDTA, and washing at 50° C. in 1×SSC and 0.1% SDS; hybridization at 50° C. in a mixed solution of 7% sodium dodecyl sulfate (SDS), 0.5 M NaPO₄, and 1 mM EDTA, and washing at 50° C. in 0.5×SSC and 0.1% SDS; hybridization at 50° C. in a mixed solution of 7% sodium dodecyl sulfate (SDS), 0.5 M NaPO₄ and 1 mM EDTA, and washing at 50° C. in 0.1×SSC and 0.1% SDS; or hybridization at 50° C. in a mixed solution of 7% sodium dodecyl sulfate (SDS), 0.5 M NaPO₄, and 1 mM EDTA, and washing at 65° C. in 0.1×SSC and 0.1% SDS.

The specific hybridization conditions may also be hybridization at 65° C. in a solution of 6×SSC and 0.5% SDS, and then washing once with 2×SSC and 0.1% SDS, and with 1×SSC and 0.1% SDS, respectively.

SEQ ID NO.: 1 in the sequence list consists of 2145 deoxyribonucleotides.

A recombinant vector, a recombinant bacterium, a transgenic cell line, or an expression cassette containing any one of the encoding genes also falls within the protection scope of the present invention.

The recombinant expression vector may be specifically obtained by inserting deoxyribonucleotides 1-2145 starting from the 5′ end of SEQ ID NO.: 1 in the sequence list between BamHI and KpnI sites of pBin438.

A recombinant expression vector containing GmSIK1 gene may be constructed by using an existing plant expression vector.

The plant expression vector includes an Agrobacterium binary vector and a vector useful for microprojectile bombardment of plants, etc. The plant expression vector may further include a 3′-end non-translational region of an exogenous gene, that is, a polyadenylation signal and any other DNA fragments involved in mRNA processing or gene expression. The polyadenylation signal can guide polyadenosinic acid to be added to a 3′ end of an mRNA precursor, for example, non-translational regions transcribed at a 3′ end of a crown gall tumor inducing (Ti) plasmid gene of Agrobacterium tumefaciens (e.g. nopaline synthetase Nos gene), and a plant gene (e.g. soybean storage-protein gene) both have similar functions.

For convenience of identification and screening of transgenic plant cells or plants, the plant expression vector used may be processed, for example, by adding a gene (e.g. GUS gene, and luciferase gene) which is able to express in the plants and encodes an enzyme resulting in color change or a luminescent compound, an antibiotic-resistance marker gene (e.g. Gentamicin-resistance marker and Kanamycin-resistance marker), or a chemical agent-resistance marker gene (e.g. herbicide-resistance gene). In view of the safety of the transgenic plants, no selectable marker gene is added, and the plants are directly screened and transformed under stress.

A primer pair for amplifying a full-length or any fragment of any one of the encoding genes described above also falls within the protection scope of the present invention.

The primer pair may be specifically as shown in 1), 2), 3), or 4) below:

1) one primer having a sequence as shown in SEQ ID NO.: 7 in the sequence list, and the other primer having a sequence as shown in SEQ ID NO.: 8 in the sequence list, where the primer pair is used for amplifying a full-length gene;

2) one primer having a sequence as shown in SEQ ID NO.: 9 in the sequence list, and the other primer having a sequence as shown in SEQ ID NO.: 10 in the sequence list, where the primer pair is used for amplifying part of fragments of a gene;

3) one primer having a sequence as shown in SEQ ID NO.: 3 in the sequence list, and the other primer having a sequence as shown in SEQ ID NO.: 4 in the sequence list, where the primer pair is used for amplifying part of fragments of a gene; and

4) one primer having a sequence as shown in SEQ ID NO.: 5 in the sequence list, and the other primer having a sequence as shown in SEQ ID NO.: 6 in the sequence list, where the primer pair is used for amplifying part of fragments of a gene.

The last objective of the present invention is to provide a method for cultivating a stress tolerant plant.

The method for cultivating a stress tolerant plant provided in the present invention includes introducing any one of the encoding genes described above to a plant, and cultivating the plant, to obtain a stress tolerant plant.

A use of any one of the proteins or any one of the encoding genes in cultivating stress tolerant plants also falls within the protection scope of the present invention.

In the method and use, the stress tolerance is salt tolerance and/or drought tolerance; and the plant is a dicotyledon, and preferably soybean, alfalfa, Lotus corniculatus or Arabidopsis thaliana.

It should be understood that the transformed cells, tissues, or plants include not only a final product of the transformation process, but also a transgenic filial generation.

As used herein, “polynucleotide”, “polynucleotide molecule”, “polynucleotide sequence”, “encoding sequence”, “open reading framework (ORF)” and the like include a single-strand or double-strand DNA or RNA molecule, and may include one or more prokaryotic sequences, cDNA sequences, genomic DNA sequences including exon and intron, chemically synthesized DNA and RNA sequences, as well as a sense and the corresponding anti-sense strand.

The gene of the present invention may be introduced into a host by inserting the gene of the present invention into an expression cassette, and then introducing the expression cassette into the host by using a plant expression vector, a non-pathogenic self-replicating virus or agrobacterium. The plant cells or tissues may be directly transformed by the expression vector carrying the gene of the present invention by using a Ti plasmid, an Ri plasmid or a plant virus vector, direct DNA transformation, micro-injection, electroporation, mediation by agrobacterium, and other conventional biological methods.

The stress tolerance is specifically stress tolerance to abiotic stress, for example, salt tolerance and/or drought tolerance.

In any one of the method and the use, the plant may be a monocotyledon or a dicotyledon, including, but not limited to, corn, wheat, barley, rye, sweet potatoes, beans, peas, chicory, lettuce, cabbage, cauliflower, broccoli, turnip, radish, spinach, asparagus, onion, garlic, pepper, celery, squash, pumpkin, cannabis, zucchini, apple, pear, quince, melon, plum, cherry, peach, nectarine, apricot, strawberry, grape, raspberry, blackberry, pineapple, avocado, papaya, mango, banana, soybean, tomato, sorghum, sugar cane, sugar beet, sunflower, rape, clover, tobacco, carrot, cotton, alfalfa, rice, potatoes, eggplant, cucumber, plants of Arabidopsis and woody plants such as coniferous and deciduous trees. Particularly preferred is rice, wheat, barley, corn, oat, rye, Lotus corniculatus, Arabidopsis thaliana, cucumber, tomato, poplar, lawn grass, or alfalfa.

The gene of the present invention that is transferred into the plant may be expanded in the species of the plant, or transferred to other varieties of the same species through a conventional breeding technology, especially including commercial varieties.

In the plant into which the gene of the present invention is transferred, biological synthesis of the plant stress tolerance related protein of the present invention occurs, so that the plant into which the gene of the present invention is transferred develops an improved trait.

The gene of the present invention may be modified as follows on the basis of SEQ ID NO.: 1, and then introduced into a host, to achieve a better expression effect.

1) In order to express the nucleotide sequence of the present invention in a transgenic plant, the nucleotide sequence of the present invention may be modified and optimized as desired. For example, the codon may be changed according to codon preferred by the receptor plant while the amino acid encoded by the nucleotide sequence of the present invention is maintained, so as to fit the preference of the plant. Moreover, in the optimization process, it is preferred that a certain GC content is maintained in the optimized encoding sequence, so as to best achieve the high-level expression of the gene introduced into the plant, where the GC content may be 35%, preferably 45% or more, more preferably 50% or more, and most preferably about 60% or more.

2) In order to effectively initiate the translation, a gene sequence adjacent to starting methionine may be modified, for example, by using a sequence known to be effective in the plant.

3) The gene of the present invention is attached to various plant expression promoters, so as to facilitate the expression of the gene in the plant. The promoters may include constitutive, inducible, temporally regulated, developmentally regulated, chemically regulated, tissue preferred, and tissue-specific promoters. The selection of the promoter varies with the requirements for expression time and space, and also depends on target species. For example, the tissue- or organ-specific expression promoter is selected, depending on the developmental stage at which the receptor is required to be stress tolerant. Although it is evidenced that numerous promoters derived from dicotyledons are useful in monocotyledons, and vice versa, desirably, the promoters derived from dicotyledons are selected for expression in the dicotyledons, and the promoters derived from monocotyledons are selected for expression in monocotyledons.

The preferred constitutive promoter includes CaMV 35S and 19S promoters. The promoter may also be a promoter derived from several actin genes expressed in most types of cells. Another preferred constitutive promoter is ubiquitin promoter.

The promoter may also be a promoter that directs expression in roots, pith, leaves, or pollen, that is, tissue-specific promoter, for example, cotton ribulose bisphosphate carboxylase-oxygenase promoter (U.S. Pat. No. 6,040,504), rice sucrose synthase promoter (U.S. Pat. No. 5,604,121), and cestrum yellow leaf curling virus promoter (WO 01/73087).

The chemically inducible promoter may be Rab29A promoter (US Patent No. US 5,614,395).

4) The gene of the present invention is attached to a suitable transcription terminator, to improve the expression efficiency of the gene of the present invention, for example, tml derived from CaMV, and E9 derived from rbcS. Any available terminator that is known to function in the plant can be attached to the gene of the present invention.

5) An enhancer sequence may be introduced into the gene of the present invention, for example, an intron sequence (for example, derived from Adhl and bronzel) and a virus leader sequence (for example, derived from TMV, MCMV, and AMV).

In practical operation, the gene of the present invention may also be subjected to cell targeting positioning, by using the technologies in the prior art. For example, before positioning, a target gene sequence derived from a target organelle is fused to the gene sequence of the present invention, and then introduced into a plant cell.

A starting vector in the recombinant vector may be selected according to the transformation technology used and the properties of the target plant species. The selection may be embodied by the selection of a resistant marker in the vector. For some target species, different antibiotic- or herbicide-selectable markers may be preferentially selected. The selectable markers used in transformation generally include nptll gene imparting resistance to Kanamycin and related antibiotics, bar gene imparting resistance to herbicide phosphinothricin, hph gene imparting resistance to antibiotic Hygromycin, dhfr gene imparting resistance to methatrexate, EPSPS gene imparting resistance to glyphosate, and mannose-6-phosphate isomerase gene that provides ability to metabolize mannose.

In another preferred embodiment, the nucleotide sequence of the present invention is directly transformed into a plastid genome. Main advantages of plastid transformation are that the plastid generally can express bacterial genes without substantial modification, and the plastid can express multiple open reading frameworks (ORFS) under control of a single promoter. Plastid expression of the gene inserted into all of the thousands of copies of the circular plastid genome existing in each plant cell through homologous recombination takes advantage of the predominance of the copy number with respect to the nuclear expression genes, so that the expression level can easily exceed 10% of the total soluble plant proteins. The gene of the present invention is inserted into a plastid targeting vector, and then transformed into an expected plant host plastid genome. In this way, a plant that is homogeneous with respect to the plastid genome containing the nucleotide sequence of the present invention is obtained, which has the ability to express the nucleotide sequence at a high level.

Experiments prove that the protein and the encoding gene thereof according to the present invention can significantly improve the salt tolerance and drought tolerance of the plants, and the stress resistance of the plants into which the gene of the present invention is transferred is obviously higher than that of those into which the gene of the present invention is not transferred. For example, after high salt or drought treatment, the recovery of the transgenic Lotus corniculatus is obviously better than that of the nontransgenic Lotus corniculatus, and the survival rate is also obviously higher than in the control group. The stress tolerance related protein and the encoding gene thereof according to the present invention are of great significance for cultivating stress tolerant plant varieties, and especially for cultivating varieties tolerant to abiotic stress such as drought and/or salt, so as to improve the yield of crops. Therefore, the protein and the encoding gene thereof according to the present invention have a broad application prospect in the field of cultivation of stress tolerant plants, and crop breeding.

### BRIEF DESCRIPTION OF THE DRAWINGS

FIG. 1 shows electrophoresis results of fragments obtained by 5′-RACE (left) and 3′-RACE (right);

FIG. 2 is schematic structure diagram of a GmSIK1 protein;

FIG. 3 shows expression patterns of the GmSIK1 gene in different tissues and organs;

FIG. 4 shows expression patterns of the GmSIK1 gene under different stresses;

FIG. 5 is a schematic diagram of plant over-expression vector pBin438-GMS/K1;

FIG. 6 shows RT-PCR detection of transgenic lotus corniculatus L. with high GmSIK1 expression level;

FIG. 7 shows growth of GmSIK1-transgenic lotus corniculatus L. under normal conditions;

FIG. 8 shows growth and survival rate of GmSIK1-transgenic lotus corniculatus L. under salt stress; and

FIG. 9 shows growth and survival rate of GmSIK1-transgenic lotus corniculatus L. under drought stress

### DETAILED DESCRIPTION OF THE EMBODIMENTS

Experimental methods used in the following embodiments are all conventional methods, if there is no special instruction.

Materials and reagents used in the following embodiments are all commercially available, if there is no special instruction.

Seeds of soybean [Glycine max (L.) Merr] Nannong 1138-2: acquisition route: plant, present; direct source: non-collection method; acquisition time: June, 1998; name of the provider: National Center for Soybean Improvement, Nanjing Agricultural University; contact of the provider: 025-84395331; and address of the provider: Weigang No. 1, Xuanwu District, Nanjing, China. The original source is unclear.

Seeds of soybean [Glycine max (L.) Merr] Nannong 1138-2 (Luo Qingyun, Yu Bingjun, Liu Youliang, Effect of NaCl on the Growth, K⁺,Na⁺ and Cl⁻ Distribution in Seedlings of 6 Soybean Cultivars (Glycine max L. Merrill), SOYBEAN SCIENCE, 2001 20(3)) (Institute of Genetics and Developmental Biology, Chinese Academy of Sciences);

pBIN438 vector (Li Taiyuan, Tian Yingchuan, Qin Xiaofeng, et al. Study on High-efficiency Insect-resistant Transgenic Tobacco, Science in China (Series B), 1994, 24(3): 276-282) (Institute of Genetics and Developmental Biology, Chinese Academy of Sciences); and

Agrobacterium GV3101 strain (Xu Pinsan, Liu Lan, Xia Xiuying, Establishment of Agrobacterium-mediated Genetic Transformation System for Lilium Siberia, Journal of Dalian University of Technology, 2008 48(5)) (Institute of Genetics and Developmental Biology, Chinese Academy of Sciences);

Seeds of Lotus corniculatus L. Leo (Guan Ning, Wang Yongxin, Li Cong, Miao Lihong, Zhang Bo, Construction of Plant Expression Vectors for Sulphur-amino Acid Gene and Transformation to Lotus corniculatus L., Molecular Plant Breeding, 2009, Vol. 7, No. 2, Pages 257-263)(Institute of Genetics and Developmental Biology, Chinese Academy of Sciences).

Embodiment 1 Screening of soybean stress tolerance related receptor kinase gene GmSIK1 and cloning of cDNA thereof

I Screening of GmSIK1

The completion of soybean genome sequencing provides favorable conditions for studying and developing new stress tolerant genes. EST-clustering was performed on 30,000 EST sequences determined by the inventors and 280,000 sequences downloaded from GenBank, to obtain 56,147 unigenes, including 32,278 Contigs and 23,869 singletons. On the basis of existing annotations of unigenes of Arabidopsis thaliana, functions of unigenes of soybean were classified and annotated. The known soybean receptor-like kinase sequence was aligned with soybean unigenes, to obtain 486 soybean receptor-like kinase candidate genes. 338 gene fragments with complete sequences were selected and used to design an RT-PCR primer, and responses under stresses of salt, drought, cold and ABA processing were analyzed. A type of gene fragment was obtained through screening, of which the expression was significantly induced by salt stresses.

II Acquisition of GmSIK1

1. Acquisition of GmSIK1 Spliced Sequences

A gene fragment obtained through EST splicing has a length of 1315 bp, and gene nested specific primers designed for 5′-RACE and 3′-RACE according to the 1315 by sequence are as follows:

Gene specific primers for 5′-RACE:
Primer 1: GmSIKl-GSP:
(Sequence 3)
5′-TAGACGTGTAGCTCAAAGAGCGCACCAA-3′;
and
Primer 2: GmSKl-NGSP:
(sequence 4)
5′-TGT GGG AAA GGC GTA GCC AGG GAT GTTT-3′.
Gene specific primers for 3′-RACE:
Primer 3: GmSIK1-GSP:
(Sequence 5)
5′-CCT CAC ACA GAG CAC TCA AGC CAA CAT TTC-3′;
and
Primer 4: GmSIK1-NGSP:
(Sequence 6)
5′-ATT CAC ATG GAA GAG GTC TCG GAA GAGG-3′.

Seeds of soybean [Glycine max (L.) Merr] Nannong 1138-2 were placed in a culture dish, grown in a culturing room for 2-3 weeks, lg fresh leaves were collected, ground in liquid nitrogen, suspended in 4mol/L guanidine thiocyanate, and extracted with acidic phenol and chloroform, and anhydrous ethanol was added to the supernatant to precipitate total RNA.

The RNA is used to synthesize cDNA by using a reverse transcriptase. Unknown sequences at 5′ terminal and 3′ terminal of the gene fragment were cloned by using the RACE method, and the specific operations were carried out according to the SMARTRACE (CLONTECH) kit. The specific steps are as follows.

1) Reverse Transcription

5′-RACE was: 1 μL soybean total RNA, 1 μL each of

Primer 1 and Primer 2, 1 μL SMART II oligo primer (10 μM) and 2 μL sterile water. 3′-RACE was: 1 μL soybean total RNA, 1 μL each of Primer 3 and Primer 4 and 3 μL sterile water. After being mixed uniformly, the mixture was heated at 70° C. for 2 min, and cooled on ice for 2 min. 2 μL 5× buffer, 1 μL DTT (20 M), 1 μL dNTP (10 M) and 1 μL powerscript reverse transcriptase were added. Reverse transcription was carried out at 42° C. for 90 min, and the mixture was heated at 72° C. for 2 min to terminate the reaction.

2) PCR Amplification

A universal primer mixture UPM and a gene specific primer were used to perform “Touchdown” PCR amplification. In Perkin-Elmer(PE) DNA Thermal Cycler 9600, the PCR reaction was initiated, in which the conditions were: 5×(94° C., 30 s; 72° C., 3 min), 5×(94° C., 30 s; 70° C., 30 s; 72° C., 3 min), 25×(94° C., 5 s; 68° C., 10 s; 72° C., 2 min). The product of the 1^{st} amplification was diluted by 20 times and served as a template for the 2^{nd} amplification.

FIG. 1 shows electrophoresis results of fragments obtained by 5′-RACE and 3′-RACE, in which 1: marker; 2: a fragment obtained by 5′-RACE; and 3: a fragment obtained by 3′-RACE.

A single band of approximately 800 by was obtained by 5′-RACE, and a single band of approximately 350 bP was obtained by 3′-RACE. The two fragments were recovered from the gel, purified and attached to a pGEM-T Easy vector for sequencing. The tested sequence was spliced with the known sequence, to obtain a spliced sequence S.

A tail of PolyA generally exists at a mature mRNA 3′ terminal, and a PolyA sequence exists at the 3′ terminal of the spliced sequence S. Through SMART software analysis, a signal peptide exists at the 5′ terminal of the spliced sequence S. It is verified through NCBI BLAST analysis that the spliced sequence S is a full-length gene sequence.

The obtained GmSIK1 sequence is 2275 bp, in which the 5′ non-coding region is 43 bp, the 3′ non-coding region is 87 bp, and the coding region consists of 2145 deoxyribonucleotides. A protein coded by the GmSIK1 sequence contains 714 amino acids.

2. Cloning of GmSIK1 cDNA

A pair of specific primers was designed according to the full-length gene sequence: primer GmSIK1FL5′-CGC GGA TCC ATG TGT GTC TTA CTT CCT TCC-3′ (Sequence 7) and GmSIK1FR 5′-CGG GGT ACC TCAAGA GTT GTT CTC CAG TG-3′ (sequence 8), with which a 2145 by GmSIK1 full-length gene was obtained through amplification.

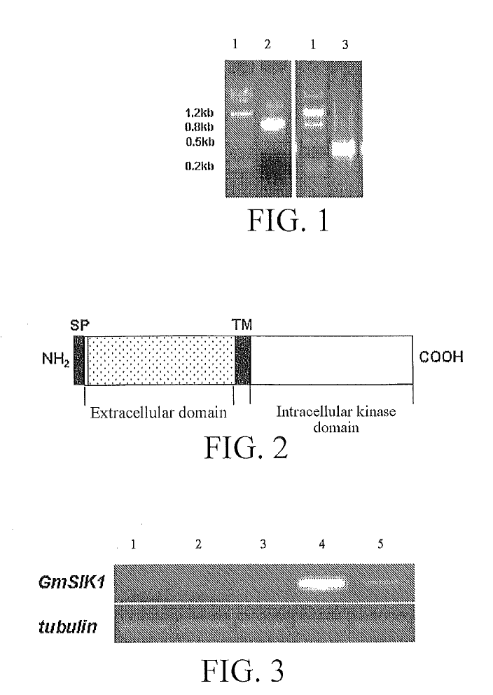

With cDNA of a leaf of seedling of soybean Nannong 1138-2 as a template, PCR amplification was performed, and the PCR product was subjected to 0.8% agarose gel electrophoresis detection, to obtain a band having a molecular weight of approximately 2.7 kb, which was consistent with the expected results. The fragment was recovered by using an agarose gel recovery kit (TIANGEN). The recovered fragment was attached to a pGEM-T Easy (Promega), following the Cohen method (Proc Natl Acad Sci, 69:2110), the attachment product was transformed into an escherichia coli DH5a competent cell, and positive clones were screened according to a carbenicillin resistant marker on the pGEM-T Easy vector, to obtain a recombinant plasmid containing the recovered fragment. With T7 and SP6 promoter sequences on the recombinant plasmid vector as primers, the nucleotide sequence was determined. The sequencing results show that the nucleotide sequence of the amplified gene is represented by SEQ ID NO.: 1 in the sequence list, and the gene consists of 2145 deoxyribonucleotides, and an amino acid sequence of a protein coded by the gene is represented by SEQ ID NO.: 2 in the sequence list. The gene represented by SEQ ID NO.: 1 is denominated as GmSIK1, the protein represented by SEQ ID NO.: 2 is denominated as GmSIK1, and the pGEM-T Easy vector containing the gene represented by SEQ ID NO.: 1 is denominated as pTE-GmSIK1. The GmSIK1 protein consists of 714 amino acids; the domain of the protein is predicted by using the SMART software, and it is indicated that the GmSIK1 protein contains a signal peptide (amino acid residues 1-33 starting from an N-terminus), 3 transmembrane regions (amino acid residues 5-27, 294-313, and 333-355 respectively, starting from an N-terminus) and an intracellular kinase domain (amino acid residues 392-662 starting from an N-terminus), thus having typical features of the structure of the receptor-like kinases. The receptor-like kinase family may be classified into different subfamilies according to the evolutionary relationship of the kinase domains, and according to this classification method, GmSIK1 belongs to the LRK-10L subfamily, which has no known extracellular domain (FIG. 2).

Embodiment 2 Expression patterns of GmSIK1 in different tissues and organs

RNAs of roots, stems and leaves of two-week old seedlings of soybean Nannong 1138-2 and flowers and pods of field soybean Nannong 1138-2 were respectively extracted, and expression levels of GmSIK1 were analyzed through RT-PCR, in which tubulin gene was used as a control, and primers were: 5′-AAC CTC CTC CTC ATC GTA CT-3′ (SEQ ID NO.: 9) and 5′-GAC AGC ATC AGC CAT GTT CA-3′ (SEQ ID NO.: 10).

Expression patterns of GmSIK1 gene in different tissues and organs are shown in FIG. 3, in which 1: roots; 2: stems; 3: flowers; 4: leaves; 5: pods.

The results show that GmSIK1 has a high expression level in leaves, and very low expression levels in other tissues and organs.

Embodiment 3 Expression patterns of soybean GmSIK1 gene under different stresses

Soybean Nannong 1138-2 [Glycine max (L.) Merr] was seeded in a flower pot filled with vermiculite, after two-week growth (with 5-6 true leaves grown), seedlings were taken and subjected to the following stress treatments.

1) Abscisic acid (ABA) treatment: completely immersing roots into a 100 μM ABA solution; 2) salicylic acid (SA) treatment: spraying 2 mM salicylic acid on leaves of seedlings; 3) drought stress treatment: placing the soybean seedlings in air at room temperature to be subjected to drought stress; 4) low temperature (4° C.) stress treatment: completely immersing the roots in an aqueous solution, and keeping the aqueous solution at 4° C.; and 5) salt stress treatment: completely immersing the roots in a 200 mM NaCl solution. 6) Control: completely immersing the roots in an aqueous solution, and keeping the aqueous solution at room temperature.

After the foregoing treatments, the seedlings were cultured under illumination, and leaves were taken at 0 h, 1 h, 3 h, 6 h, 12 h, and 24 h after the treatment, and RNAs were extracted. With the tubulin gene as a control, the expression level of GmSIK1 was analyzed through RT-PCR.

Primers:
(Sequence 9)
5′-AAC CTC CTC CTC ATC GTA CT-3′
and
(SEQ ID NO.: 10)
5′-GAC AGC ATC AGC CAT GTT CA-3′.

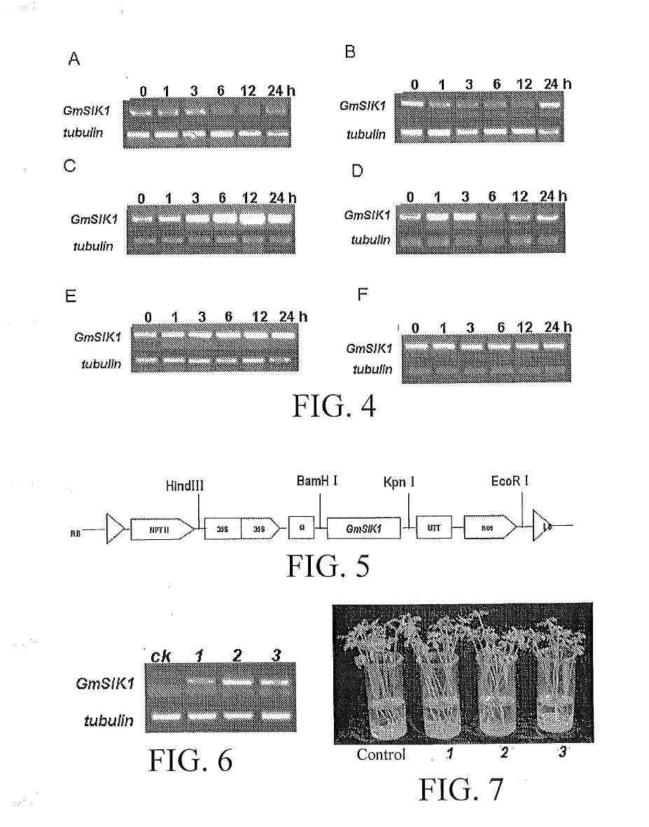

Expression patterns of the GmSIK1 gene under different stresses were shown in FIG. 4, in which A: abscisic acid treatment; B: salicylic acid (SA) treatment; C: drought stress treatment; D: low temperature stress treatment; E: salt stress treatment; F: control.

The results show that: 1) ABA treatment: the expression level of GmSIK1 reaches a peak at 3 h, and then declines; 2) salicylic acid treatment: the expression level of GmSIK1 rises at 1 h, then declines and rises sharply to a peak at 24 h; 3) drought stress: the expression level of GmSIK1 rises significantly at 1 h of stress, reaches a peak at 12 h, and slightly declines at 24 h; 4) low temperature stress: the expression level of GmSIK1 rises at 1 hr and reaches a maximum value at 3 h, then declines, and rises again at 24 h; 5) salt stress: the expression level of GmSIK1 starts to significantly and gradually rise at 1 h of stress, reaches a peak value at 12 h, and slightly declines at 24 h; and 6) the expression level of GmSIK1 has almost no variation in the control group with water added.

Embodiment 4 Acquisition of GmSIK1-transgenic plant and stress tolerance identification

I Construction of GmSIK1 expression vector pBin438-GmSIK1

The GmSIK1 gene was cleaved from the recombinant vector PTE-GmSIK1 with restriction endonucleases BamH I and Kpn I, and forward inserted into the BamH I and Kpn I sites of the pBIN438 vector, so that a target gene was expressed under the induction of the double CAMV35S promoter and the Q sequence. The recombinant plasmid obtained by correctly inserting the GmSIK1 gene is denominated as pBIN438-GmSIK1, and the structure of the recombinant vector is shown in FIG. 5. The results of cleavage and sequencing verification show that the insertion direction and the sequence of the GmSIK1 gene in the constructed recombinant vector pBIN438-GMS/K1 are correct.

II Acquisition of GmSIK1-transgenic lotus corniculatus L. and Stress tolerance Identification 1. Acquisition of GmSIK1-transgenic lotus corniculatus L.

pBIN438-GmSIK1 was transferred into an Agrobacterium GV3101 strain through electro-transformation. The Agrobacterium with the recombinant vector pBIN438-GmSIK1 transferred was transformed into lotus corniculatus L. Leo (lotus corniculatus L.).

The transformation method includes: stem nodes of well-gown aseptic seedlings were infected with Agrobacterium for 20-30 min, transferred into an induction medium and cultured at 25° C. in the dark for 3 days; then transferred into an MS screening medium and cultured for 30 days; and then transferred into an MS screening medium containing 80 mg/mL kanamycin; the resistant plants obtained through screening were rooted under induction, and transferred into a greenhouse, and transferred into a farm when the appropriate season arrived. In total, 70 resistant strains were obtained through screening.

The MS medium is available from Qingdao Hope Bio-Technology Co., Ltd. in Qingdao Science and Technology Town, Trademark: Qingdao Hope, Model: HB8469.

Composition of the induction medium is as follows. Saccharose is added to the MS medium to a concentration of 30 g/L, and naphthylacetic acid (NAA) and 6-benzylaminopurine (BAP) are added, so that the concentration of NAA in the induction medium is 0.5 mg/L, and the concentration of BAP in the induction medium is 0.5 mg/L.

The composition of the MS screening medium is as follows. BAP, cefotaxime and kanamycin are added into the MS medium; the concentration of BAP in the MS screening medium is 0.2 mg/L, the concentration of cefotaxime in the MS screening medium is 500 mg/L, and the concentration of the screening agent kanamycin in the MS screening medium is 50 mg/L.

Meanwhile, the empty vector-transformed pBIN438 was transformed into lotus corniculatus L.to serve as control strain (denoted as (CK)), and the method is the same as the preparation of the transgenic plant.

The 70 resistant strains were identified through RT-PCR (these 70 strains were T₁ generation), and the RT-PCR identification method is as follows. Leaves of each plant were taken, and RNAs were extracted, and then subjected to RT-PCR. The primers were:

(SEQ ID NO.: 9)
5′-AAC CTC CTC CTC ATC GTA CT-3′
(SEQ ID NO.: 10)
5′-GAC AGC ATC AGC CAT GTT CA-3′.

As a result, it was identified that over-expression of the GmSIK1 gene exists in 7 strains among the 70 resistant strains, and 3 stains (respectively denoted as 1#strain, 2#strain and 3#strain) with high but different expression levels were further screened (these 3 strains were the T1 generation) for further function tests. The RT-PCR identification results of these 3 strains are shown in FIG. 6 (CK represents the control, and 1, 2 and 3 respectively represent 1#strain, 2#strain and 3#strain). Different degrees of heterologous over-expression of the GmSIK1 gene exist in the selected 3 transgenic plants, and no expression of the GmSIK1 gene exists in the empty vector-transformed control plants.

2. Growth of GmSIK1-Transgenic Plant under Normal Conditions

From 3 transgenic plant strains (1#strain, 2#strain and 3#strain) and an empty vector-transformed control plant (CK) growing in a field, several newly grown lateral branches having a length of 13-15 cm were taken and cultured with water in a bottle, and radicles were grown after seven days.

The growth of an 8-day plant is shown in FIG. 7. The results show that, under normal conditions, no significant difference of growth exists between the 3 transgenic plant strains and the empty vector-transformed control plant.

Method for culturing lotus corniculatus L with water is: clipping lateral branches on similar positions, and immersing in water for 7 days, to grow radicles.

3. Stress tolerance detection of GmSIK1-transgenic plant

In the following, the growth of 1#strain, 2#strain and 3#strain and the empty vector-transformed control plant under stresses are respectively tested.

1) Salt Tolerance Test

From 3 transgenic plant strains (1#strain, 2#strain and 3#strain) and an empty vector-transformed control plant (CK) growing in a field, several newly grown lateral branches having a length of 13-15 cm were taken and cultured with water in a bottle, and radicles were grown after seven days. The radicles were respectively transferred to a 100 mM NaCl solution, and after 24-day treatment placed in normal conditions to restore growth for 3 days. Phenotype was observed and photographs were taken, and at the same time, statistics were taken on the survival rate. The experiments were repeated three times, and 10 plants of each strain were detected each time, and the test data was an average±standard deviation of the three repeated experiments.

Calculation formula of the survival rate for each repetition: the survival rate=the number of survived plants/10 plants×100%.

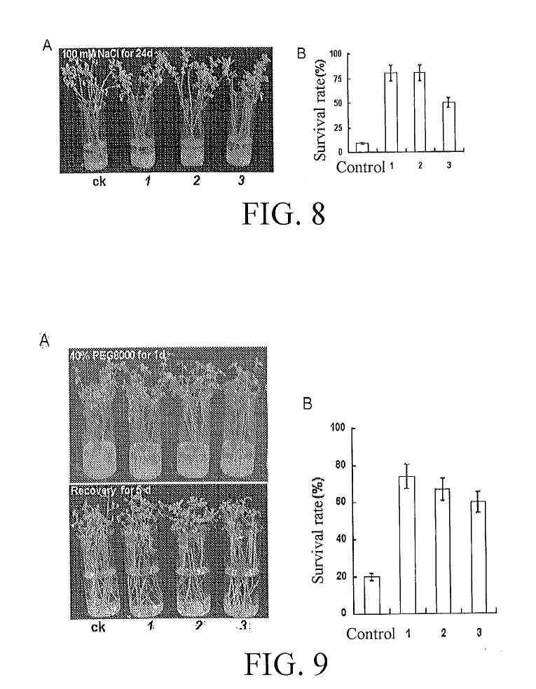

The results are shown in FIG. 8 (CK and control both represent control groups, 1, 2 and 3 respectively represent 1#strain, 2#strain and 3#strain), FIG. A shows phenotypic photos of plants after 24-day salt treatment; and FIG. B shows comparison of the survival rates of the plants.

The results show that, after 24-day NaCl treatment, most of the leaves of the control plants are withered, the top newly grown leaves are dead; while merely a small number of lateral branches of the transgenic plants are withered, and leaves of most plants get thick and yellow, but the plants are still growing, thus transgenic plants show better salt resistance; the survival rates of the GmSIK1-transgenic strains 1#strain, 2#strain and 3#strain are 80%, 80% and 52% in sequence, all are higher than 50%, while the survival rate of the control is merely approximately 10%.

2) Drought Tolerance Identification

An aqueous PEG solution was used to simulate an arid soil environment, in which PEG served as an osmosis adjustment substance, as its molecular weight is high and will not pass through the cell wall and produce a dehydration effect on the cell similar to that caused by dry soil. From 3 transgenic plant strains (1#strain, 2#strain and 3#strain) and an empty vector-transformed control plant (CK) growing in a field, several newly grown lateral branches having a length of 13-15 cm were taken and cultured with water in a bottle, and radicles were grown after seven days. The radicles were respectively immersed in a 40% PEG solution, and observed and photographed after 1 day. Water supply was restored for 5 days, and phenotype was observed and photographs were taken, and at the same time, statistics were taken on the survival rate. The experiments were repeated three times, and 10 plants of each strain were detected each time, and the test data was an average±standard deviation of the three repeated experiments.

Calculation formula of the survival rate for each repetition: the survival rate=the number of survived plants/10 plants×100%.

The results are shown in FIG. 9 (CK and control both represent the control groups, 1, 2 and 3 respectively represent 1#strain, 2#strain and 3#strain), FIG. A shows phenotypic photos of plants after 1-day PEG treatment (upper) and restoring water supply for 5 days (lower); and FIG. B shows comparison of the survival rates of the plants.

The results show that after PFG treatment, all of the material leaves are dehydrated and wilted, and no significant difference of phenotype exists. After restoring water supply, about 80% of the control material leaves are withered due to dehydration, and the top cannot grow any more. Among the transgenic plants, most of the leaves that are dehydrated and wilted by PEG can re-absorb water and restore the normal growth. The survival rates of the three strains (1#strain, 2#strain and 3#strain) are approximately 75%, 68% and 60% respectively, while the survival rate of the control is merely 20%.

The experiments show that, the heterologous expression of GmSIK1 in lotus corniculatus L. improves the drought resistance and the salt resistance of the transgenic plants. GmSIK1 is involved in tolerance signal transduction of plants.

1. A proteinselected from the group consisting off:
(a) a protein consisting of amino acid residues 34-714 starting from an N-terminus of an amino acid sequence as shown in SEQ ID NO: 2;
(b) a protein consisting of the amino acid sequence as shown in SEQ ID NO: 2;
(c) a protein related to plant stress tolerance and derived from the protein of (a) by substituting and/or deleting and/or adding one or more amino acid residues in the amino acid sequence as shown in SEQ ID NO: 2; and
(d) a protein related to plant stress tolerance and consisting of an amino acid sequence that is at least 75%, at least 80%, at least 85%, at least 90%, at least 95%, at least 96%, at least 97%, at least 98% or at least 99% homologous with the amino acid sequence as shown in SEQ ID NO 2.

2. An encoding gene of the protein according to claim 1.

3. The encoding gene according to claim 2, wherein the encoding gene is selected from the group consisting of 1) a DNA molecule comprising the nucleotide sequence of SEQ ID NO: 1;
2) a DNA molecule that specifically hybridizes with the DNA molecule defined in 1) under stringent conditions and encodes a plant stress tolerance related protein; and
3) a DNA molecule that is at least 70%, at least 75%, at least 80%, at least 85%, at least 90%, at least 95%, at least 96%, at least 97%, at least 98% or at least 99% homologous with the DNA sequence defined in 1) and encodes a plant stress tolerance related protein.

4. A recombinant vector, a recombinant bacterium, a transgenic cell line, or an expression kit comprising the encoding gene according to claim 2.

5. A primer pair for amplifying a full-length or any fragment of the encoding gene according to claim 2.

6. The primer pair according to claim 5, wherein the primer pair is selected from the group consisting of:
1) one primer comprising the nucleotide sequence of SEQ ID NO: 7, and another primer comprising the nucleotide sequence of SEQ ID NO: 8;
2) one primer comprising the nucleotide sequence of SEQ ID NO: 9, and another primer comprising the nucleotide sequence of SEQ ID NO: 10;
3) one primer [[having a sequence as shown in]]comprising the nucleotide sequence of SEQ ID NO: 3, and another primer comprising the nucleotide sequence of SEQ ID NO: 4; and
4) one primer comprising the nucleotide sequence of SEQ ID NO: 5, and another primer comprising the nucleotide sequence of SEQ ID NO: 6.

7. A method for cultivating a stress tolerant plant, comprising introducing the encoding gene according to claim 2 to a plant, and cultivating the plant, to obtain a stress tolerant plant.

8. The method according to claim 7, wherein the stress tolerance is salt tolerance and/or drought tolerance.

9. The method according to claim 7, wherein the plant is a dicotyledon, and preferably soybean, alfalfa, Lotus corniculatus or Arabidopsis thaliana.

10. (canceled)


SEQUENCE LISTING (10 sequences)

SEQ ID NO: 1 - DNA, 2145 bp, Glycine max
atgtgtgtcttacttccttccttcctcttttcagaatcaatggttacctctctggctatcataattcttgtgctgctattccaacaaacttgccttgccaagcagcaccaccaccacccaccctgttattcttcatgtggcgaaattcacaacataacgtatccatttcgactaaaaggcgacccaatcggctgcggcgatcaagattacgaattagattgcgttgaaaacgtgacagtaatgactctattttcagggaaattccacgtgcaggaaataaattacaagaggtacgagatccggttgaccgatgctggtgtggtggaggacattgcttgctcaattcctcgttatttctttaatctctacaatttcacttacatcggtggtcttaactattctgcatatggtccgttaagtttctcggggtatcctcagtatgcggtgtttctaaactgcactaccccagtcaccgatgatcctcgatacgtagctgtgaaggtcaacggtaattgtgattcgagttccgtaggccatatctatgccattctggtcacagacgatgatgatcgctttgggtttacactgacggacatcaaggttgggtgccgattgaaggttgctacatttgcaaactggaaatataataacagggaggttaacgtttcttattctgatatcaacaagtggctgcaccacggaatttggatgtcttggttctttccagttatttgtagggaccgatgcggaaagggtgtgagttgctacatcgatcaaaccacgcgagaaactcgatgtgcagatagctactgccaaatctttggcattcaaacatacaagtgcggaattcgtaatcagattcttgaatatatgcggcaaattcttggctacacagtaggttatcttaaaagcgttataatgggatatgtaaatagaataagatatcgcgagcaactaacaagctgggatagtgaagcagaattctttgaacaaaatgctatagcaatattcctggcaaccagattgctatttggaatcacacttttactaatgctatatatctatatgtggcgacgtagacattattcaatgtatgaaaacattgaaatttttttgctagatagcaatctaaatcctattaggtatgaatacagagaaataaaaaaaatgaccaaagatttcaaagtgaaattgggtgaaggaggtttcggctctgtatacaaaggaaaacttcgaagcgggctagatgtggcaataaagatgttgaccaaatccaaaactagaggacaagattttatcagtgaagtggctacaattggaagaatacatcacgtgaatgtggtgcgtcttattggatattgtgctgaaggagaaaagcatgctctagtttatgaattcatgcctaatggttcattggataaatacattttctctaaagaagaaagtgtctccttaagttatgacaaaacatatgaaatatgtcttggcatagctcgtgggattgcttatttgcatcaagattgtgatgtgcaaattctacattttgatatcaagcctcataatattcttctagatgataacttcattccaaaggtttcggattttggacttgcaaagctatatcctatcaaagataagtcaattattttaactggactaagaggaacttttggttacatggctccagaattattctacaaaaatattggtggagtgtcatataaggcagacgtctatagctttggaatgcttttgatggaaatgggaagtagaaggaggaactcaaatcctcacacagagcactcaagccaacatttcttccccttttggatctatgatcattttatggaagaaaaggatattcacatggaagaggtctcggaagaggataagattctagtgaaaaagatgttcatagtttccctttggtgtatacaattaaaaccaaatgatcgtccttcaatgaagaaggttgtagagatgcttgaaggaaaggttgaaaatattgatatgcctccaaagcctgttttttatccccatgaaacaacaatcgactctgatcaagcatcatggagtgattcaactagttcttgcaagaacattgacaaaaccaagtccaatttttcactggagaacaactcttga

SEQ ID NO: 2 - protein, 714 aa, Glycine max
MCVLLPSFLFSESMVTSLAIIILVLLFQQTCLAKQHHHHPPCYSSCGEIHNITYPFRLKGDPIGCGDQDYELDCVENVTVMTLFSGKFHVQEINYKRYEIRLTDAGVVEDIACSIPRYFFNLYNFTYIGGLNYSAYGPLSFSGYPQYAVFLNCTTPVTDDPRYVAVKVNGNCDSSSVGHIYAILVTDDDDRFGFTLTDIKVGCRLKVATFANWKYNNREVNVSYSDINKWLHHGIWMSWFFPVICRDRCGKGVSCYIDQTTRETRCADSYCQIFGIQTYKCGIRNQILEYMRQILGYTVGYLKSVIMGYVNRIRYREQLTSWDSEAEFFEQNAIAIFLATRLLFGITLLLMLYIYMWRRRHYSMYENIEIFLLDSNLNPIRYEYREIKKMTKDFKVKLGEGGFGSVYKGKLRSGLDVAIKMLTKSKTRGQDFISEVATIGRIHHVNVVRLIGYCAEGEKHALVYEFMPNGSLDKYIFSKEESVSLSYDKTYEICLGIARGIAYLHQDCDVQILHFDIKPHNILLDDNFIPKVSDFGLAKLYPIKDKSIILTGLRGTFGYMAPELFYKNIGGVSYKADVYSFGMLLMEMGSRRRNSNPHTEHSSQHFFPFWIYDHFMEEKDIHMEEVSEEDKILVKKMFIVSLWCIQLKPNDRPSMKKVVEMLEGKVENIDMPPKPVFYPHETTIDSDQASWSDSTSSCKNIDKTKSNFSLENNS

SEQ ID NO: 3 - DNA, 28 bp, Artificial
  RACE primer
tagacgtgtagctcaaagagcgcaccaa

SEQ ID NO: 4 - DNA, 28 bp, Artificial
  RACE primer
tgtgggaaaggcgtagccagggatgttt

SEQ ID NO: 5 - DNA, 30 bp, Artificial
  RACE primer
cctcacacagagcactcaagccaacatttc

SEQ ID NO: 6 - DNA, 28 bp, Artificial
  RACE primer
attcacatggaagaggtctcggaagagg

SEQ ID NO: 7 - DNA, 30 bp, Artificial
  PCR primer
cgcggatccatgtgtgtcttacttccttcc

SEQ ID NO: 8 - DNA, 29 bp, Artificial
  PCR primer
cggggtacctcaagagttgttctccagtg

SEQ ID NO: 9 - DNA, 20 bp, Artificial
  RT-PCR primer
aacctcctcctcatcgtact

SEQ ID NO: 10 - DNA, 20 bp, Artificial
  RT-PCR primer
gacagcatcagccatgttca